SEQUENCE LISTING (41 sequences)

SEQ ID NO: 1 - protein, 735 aa, adeno-associated virus 2 [sequence omitted]

SEQ ID NO: 2 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSTTHLSPPQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSELEEMNNKQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSPSSPAPAQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRPETQAKPQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSTAYTPAPQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRSQRETVWKQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSVMMVGGREQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRKDPEVSEQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSKTNLDRAQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSATQPYASQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRTSAHQVGEQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRSPKGSAWEQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSPQQPPRSQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSSRTPQHKQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSPRHPVTTQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSHTTGSPAKQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRMTGKTGYEQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRQATSGFDKQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRAKEMRSEQQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 743 aa, Artificial Sequence
  Synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSAFSQSAVKQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 9 aa, Artificial Sequence
  Synthetic sequence
STTHLSPPQ

SEQ ID NO: 23 - protein, 9 aa, Artificial Sequence
  Synthetic sequence
SELEEMNNK

SEQ ID NO: 24 - protein, 9 aa, Artificial Sequence
  Synthetic sequence
SPSSPAPAQ

SEQ ID NO: 25 - protein, 9 aa, Artificial Sequence
  Synthetic sequence
RPETQAKPQ

SEQ ID NO: 26 - protein, 9 aa, Artificial Sequence
  Synthetic sequence
STAYTPAPQ

SEQ ID NO: 27 - protein, 9 aa, Artificial Sequence
  Synthetic sequence
RSQRETVWK

SEQ ID NO: 28 - protein, 9 aa, Artificial Sequence
  Synthetic sequence
SVMMVGGRE

SEQ ID NO: 29 - protein, 9 aa, Artificial Sequence
  Synthetic sequence
RKDPEVSEQ

SEQ ID NO: 30 - protein, 9 aa, Artificial Sequence
  Synthetic sequence
SKTNLDRAQ

SEQ ID NO: 31 - protein, 9 aa, Artificial Sequence
  Synthetic sequence
SATQPYASQ

SEQ ID NO: 32 - protein, 9 aa, Artificial Sequence
  Synthetic sequence
RTSAHQVGE

SEQ ID NO: 33 - protein, 9 aa, Artificial Sequence
  Synthetic sequence
RSPKGSAWE

SEQ ID NO: 34 - protein, 9 aa, Artificial Sequence
  Synthetic sequence
SPQQPPRSQ

SEQ ID NO: 35 - protein, 9 aa, Artificial Sequence
  Synthetic sequence
SSRTPQHKQ

SEQ ID NO: 36 - protein, 9 aa, Artificial Sequence
  Synthetic sequence
SPRHPVTTQ

SEQ ID NO: 37 - protein, 9 aa, Artificial Sequence
  Synthetic sequence
SHTTGSPAK

SEQ ID NO: 38 - protein, 9 aa, Artificial Sequence
  Synthetic sequence
RMTGKTGYE

SEQ ID NO: 39 - protein, 9 aa, Artificial Sequence
  Synthetic sequence
RQATSGFDK

SEQ ID NO: 40 - protein, 9 aa, Artificial Sequence
  Synthetic sequence
RAKEMRSEQ

SEQ ID NO: 41 - protein, 9 aa, Artificial Sequence
  Synthetic sequence
SAFSQSAVK

(19) United States
(12) Patent Application Publication
(10) Pub. No.: US 2024/0067678 A1
(43) Pub. Date: Feb. 29, 2024

(54) ADENO-ASSOCIATED VIRUS CAPSIDS AND VECTORS

(71) Applicant: Children's Medical Research Institute, Westmead, NSW (AU)

(72) Inventors: Adrian WESTHAUS, Westmead, NSW (AU); Leszek LISOWSKI, Westmead, NSW (AU); Marti CABANES CREUS, Bondi Beach, NSW (AU)

(73) Assignee: Children's Medical Research Institute, Westmead, NSW (AU)

(21) Appl. No.: 18/267,764

(22) PCT Filed: Dec. 16, 2021
(86) PCT No.: PCT/AU2021/051497

§ 371 Date: Jun. 15, 2023

(30) Foreign Application Priority Data
Dec. 16, 2020 (AU) 2020904687

(51) Int. Cl. C07K 14/005; C12N 7/00; C12N 15/86

(52) U.S. Cl. CPC C07K 14/005; C12N 7/00; C12N 15/86; C12N 2750/14122; C12N 2750/14141

(57) ABSTRACT

The present disclosure relates generally to adeno-associated vims (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells. The disclosure includes AAV capsid polypeptides comprising a peptide modification in variable region 8 (VR VIII), where the peptide modification comprises a 7 amino acid insertion.

This application claims priority to Australian Provisional Application No. 2020904687 entitled “Adeno-associated virus capsids and vectors” filed 16 Dec. 2020, the content of which is incorporated herein by reference in its entirety.

### FIELD OF THE DISCLOSURE

The present disclosure relates generally to adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contains one or more heterologous sequences flanked by the ITRs have been successfully used in gene therapy settings. These AAV vectors are widely used to deliver heterologous nucleic acid to cells of a subject for therapeutic purposes. One example of this is the use of AAV vectors for therapy of liver-associated diseases. However, there are a limited number of AAV vectors that efficiently transduce hepatocytes in vivo, so as to treat liver-associated diseases. There remains a need, therefore, to develop novel AAV vectors suitable for the delivery of nucleic acid to the liver.

### SUMMARY OF THE DISCLOSURE

The present disclosure is predicated in part on the identification of novel AAV capsid polypeptides. The capsid polypeptides, when present in the capsid of an AAV vector, can facilitate transduction of human hepatocytes, typically at a level that is increased or enhanced compared to AAV vectors comprising a reference AAV capsid polypeptide (e.g. the prototypic AAV2 capsid set forth in SEQ ID NO:1). The capsid polypeptides of the present disclosure are therefore particularly useful in preparing AAV vectors, and in particular, AAV vectors for therapeutic applications. Similarly, AAV vectors comprising a capsid polypeptide of the present disclosure (i.e. having a capsid comprising or consisting of a capsid polypeptide of the present disclosure) are of particular use in delivering heterologous nucleic acids to the liver of a subject, such as for the treatment of various liver-associated diseases and conditions.

In one aspect, provided is an AAV capsid polypeptide, comprising a peptide modification relative to the AAV2 capsid polypeptide set forth in SEQ ID NO:1, wherein: the peptide modification is in variable region 8 (VRVIII); the peptide modification comprises a 7 amino insertion relative to the AAV2 capsid polypeptide set forth in SEQ ID NO:1, and comprises the sequence set forth in any one of SEQ ID Nos:22-41; and the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to positions 1-735, 138-735 or 203-735 of SEQ ID NO:1.

In some examples, the peptide modification is in the region of the capsid polypeptide spanning positions 585-589, with numbering relative to SEQ ID NO:1. In one embodiment, the peptide modification comprises a 7 amino insertion after position 587 relative to the AAV2 capsid polypeptide set forth in SEQ ID NO:1. In further embodiments, the peptide modification comprises amino acid substitutions at positions 587 and 588 relative to the AAV2 capsid polypeptide set forth in SEQ ID NO:1. In still further embodiments, the AAV capsid polypeptide comprises one or more amino acid substitutions at position 585, 586, and/or 589 relative to the AAV2 capsid polypeptide set forth in SEQ ID NO:1 (e.g. R585G, G586Q and/or Q589A, relative to the AAV2 capsid polypeptide set forth in SEQ ID NO:1). In one example, the AAV capsid polypeptide comprises the sequence of amino acids set forth in any one of SEQ ID NOs:2-21, the sequence of amino acids set forth as amino acids 138-742 of any one of SEQ ID NOs:2-21, or the sequence of amino acids set forth as amino acids 203-742 of any one of SEQ ID NOs:2-21; or a sequence having at least or about 85%, 86%, 87%, 88%, 89%, 90%, 91%, 92%, 93%, 94%, 95%, 96%, 97%, 98%, or 99% sequence identity thereto.

In another aspect, provided is an AAV capsid polypeptide, comprising: a) a VP1 protein comprising the sequence of amino acids set forth in any one of SEQ ID NOs:2-21; b) a VP2 protein comprising the sequence of amino acids set forth as amino acids 138-742 of any one of SEQ ID NOs:2-21; c) a VP3 protein comprising the sequence of amino acids set forth as amino acids 203-742 of any one of SEQ ID NOs:2-21; or d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c), wherein the capsid polypeptide comprises, at positions 587-595 with numbering relative to any one of SEQ ID NOs:2-21, the peptide sequence set forth in any one of SEQ ID Nos:22-41.

Also provided are AAV vectors comprising a capsid polypeptide described above and herein. In some examples, the vector exhibits increased transduction efficiency of human hepatocytes compared to an AAV vector comprising a capsid polypeptide comprising the sequence of amino acids set forth in SEQ ID NO:1 (e.g. wherein transduction efficiency is increased by at least or about 10%, 20%, 30%, 40%, 50%, 60%, 70%, 80%, 90%, 100%, 200%, 300%, 400% or 500%). The AAV vector may further comprise a heterologous coding sequence, such as a heterologous coding sequence that encodes a peptide, polypeptide or polynucleotide (e.g. a therapeutic peptide, polypeptide or polynucleotide).

In further aspects, provided is an isolated nucleic acid molecule encoding a capsid polypeptide described herein, and a vector (e.g. a plasmid, cosmid, phage and transposon) comprising the nucleic acid molecule. Also provided is a host cell, comprising an AAV vector, nucleic acid molecule or vector described herein.

In another aspect, provided is a method for introducing a heterologous coding sequence into a host cell, comprising contacting a host cell with an AAV vector of the disclosure that comprises a heterologous nucleic acid. In some examples, the host cell is a hepatocyte. In particular examples, contacting a host cell with the AAV vector comprises administering the AAV vector to a subject. These method can be in vivo, in vitro or ex vivo In other examples, administration of the AAV vector to the subject effects treatment of a liver-associated disease or condition.

Also provided is a method for producing an AAV vector, comprising culturing a host cell comprising a nucleic acid molecule encoding a capsid polypeptide described herein, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide, wherein the capsid encapsidates the heterologous coding sequence. In some examples, the host cell is a hepatocyte.

In another aspect, provided is the use of an AAV vector described herein for the preparation of a medicament for treating a liver-associated disease or condition.

### BRIEF DESCRIPTION OF THE DRAWINGS

Embodiments of the disclosure are described herein, by way of non-limiting example only, with reference to the following drawings.

FIG. 1 is a schematic of the peptide library construction. Detailed view of the modification workflow of the Ico2 capsid. Amino acid position of Q584 and A591 using numbering from un-modified cap2 VP1. First step was the insertion of two SfiI restriction sites. Second step was the insertion of the peptide library. Seven truly randomized NNK (X1-X7) insertion as well as the full 9mer peptide including semi-random flanking amino acids are shown within the modified region.

FIG. 2 shows the results of in vivo selection of an AAV2-based peptide display library using the Replication Competent (RC) and Functional Transduction (FT) platforms in human hepatocytes. a) Selection kinetics using library selection methods indicated with RC-Ad5 selection (RC) and FT-RNA selection (FT). From left to right for RC: Library; Round 1; and Round 2. From left to right for FT: Library; Round 1 (DNA); Round 1 (RNA); and Round 2 (RNA). b) Vector entry performance (DNA NGS) of most abundant variants from RC-Ad5 selection (RC-R2; left) and FT-RNA selection (FT-R2; right). Statistics: non-parametric t-test (Mann-Whitney p-value: **** ≤0.0001) c) Vector expression performance (RNA/cDNA NGS) of most abundant variants from RC-Ad5 selection (RC-R2; left) and FT-RNA selection (FT-R2; right). Statistics: non-parametric t-test (Mann-Whitney p-value: **** ≤0.0001) d) Vector expression index (RNA reads normalized to DNA reads) of most abundant variants from RC-Ad5 selection (RC-R2; left) and FT-RNA selection (FT-R2; right). Statistics: non-parametric t-test (Mann-Whitney p-value: **** ≤0.0001) e & f) Individual performance using DNA (e) or RNA/cDNA (f) amplicon seq (Illumina) of most abundant variants from RC-Ad5 selection (RC) and FT-RNA selection (FT) compared to AAV2 (lower dotted line) and NP59 (upper dotted line). Experiments were performed in 4 humanized mice which had engrafted human hepatocytes from either an adult male (circles) or an infant female (triangles). Statistics are shown for each variant with a higher average performance compared to NP59. Statistics: Nonparametric t-test (Mann-Whitney p-value: * ≤0.05).

FIG. 3 shows AAV2-based peptide variants' performance in human hepatocytes in FRG mice. The graph shows the expression index of individual capsid variants compared to AAV2 (lower dotted line) and NP59 (upper dotted line). Experiments were performed in n=4 humanized mice which had engrafted human hepatocytes from either an adult male (circles) or an infant female (triangles). Statistics are shown for each variant with a higher average expression index compared to NP59. Statistics: Non-parametric t test (Mann-Whitney p-value: * ≤0.05).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or “AAV vector” refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic, bioengineered or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the bioengineered DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, the term “transduction” refers to the ability of an AAV vector to enter one or more particular cell types and transfer the DNA contained within the AAV vector into the cell. Transduction can be assessed by measuring the amount of AAV DNA or RNA expressed from the AAV DNA in a cell or population of cells, and/or by assessing the number of cells in a population that contain AAV DNA or RNA expressed from the DNA. Where the presence or amount of RNA is assessed, the type of transduction assessed is referred to herein as “functional transduction”, i.e. the ability of the AAV to transfer DNA to the cell and have that DNA expressed. “Transduction efficiency” is a measure of the level of transduction from a starting amount of AAV vector (e.g. the starting amount of vector being injected in vivo or applied to cells in vitro), and can be quantitative or qualitative, and/or with reference to a particular control, e.g. a prototypic AAV vector. For example, if a candidate AAV vector transduces twice as many cells as a control vector and/or the amount of AAV DNA per cell from transduction with the candidate AAV vector is twice that of transduction with the control vector, where the starting amount of each vector was the same (i.e. the amount of each vector injected into a subject or applied to cells was the same), it can be said that the transduction efficiency of the candidate AAV vector is 200% greater than, or is twice that of, the transduction efficiency of the control vector.

The phrase “numbering relative to” a sequence, such as SEQ ID NO:1, means that the numbering of the amino acid position being referred to is as shown in the sequence, e.g. SEQ ID NO:1. It will be appreciated that the sequence is simply a reference sequence, and that the same amino acid residue or position may correspond to a different number in a different sequence, such as if the different sequence is a truncated form or is a sequence that has insertions or deletions compared to the reference sequence. To identify corresponding positions or residues in different sequences, sequences of related or variant polypeptides are aligned by any method known to those of skill in the art. Such methods typically maximize matches (e.g. identical nucleotides or amino acids at positions), and include methods such as using manual alignments and by using the numerous alignment programs available (for example, BLASTP, ClustlW, ClustlW2, EMBOSS, LALIGN, Kalign, etc.) and others known to those of skill in the art. By aligning the sequences of polypeptides, one skilled in the art can identify corresponding positions. For example, by aligning the prototypic AAV2 capsid polypeptide set forth in SEQ ID NO:1 with another AAV capsid polypeptide, such as the AAV2-RC01 capsid set forth in SEQ ID NO:2, one of skill in the art can identify regions or amino acids residues within AAV2-RC01 that correspond to various regions or residues in the AAV2 polypeptide set forth in SEQ ID NO:1. For example, the leucine at position 735 of SEQ ID NO:1 corresponds to the leucine at position 742 of SEQ ID NO:2.

As used herein, “corresponding nucleotides” or “corresponding amino acid residues” or grammatical variations thereof refer to nucleotides or amino acids that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc) and others known to those of skill in the art. By aligning the sequences of polynucleotides or polypeptides, one skilled in the art can identify corresponding nucleotides or amino acids. For example, by aligning the prototypic AAV2 capsid polypeptide set forth in SEQ ID NO:1 with another AAV capsid polypeptide, such as the variant set forth in SEQ ID NO:2, one of skill in the art can identify regions or amino acids residues within the other AAV polypeptide that correspond to various regions or residues in the AAV polypeptide set forth in SEQ ID NO:1, e.g. the asparagine at position 734 of SEQ ID NO:1 corresponds to the asparagine at position 741 of SEQ ID NO:2.

The term “peptide modification” refers to a modification in a polypeptide that involves two or more contiguous amino acids (i.e. that involves a peptide within the polypeptide). The peptide modification can include amino acid insertions, deletions and/or substitutions relative to a reference polypeptide. For example, an exemplary peptide modification of the present disclosure comprises 9 consecutive amino acid residues, wherein 7 of those residues are insertions relative to the prototypic AAV2 capsid set forth in SEQ ID NO:1, and 2 of those residues are amino acid substitutions relative to the prototypic AAV2 capsid set forth in SEQ ID NO:1.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “host cell” refers to a cell, such as a mammalian cell, that has introduced into it the exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

It will be appreciated that the above described terms and associated definitions are used for the purpose of explanation only and are not intended to be limiting.

TABLE 1
Description of Sequences

| SEQ ID NO | Sequence description |
|---|---|
| 1 | AAV2 prototypic capsid (VP1 protein) |
| 2 | AAV2-RC01 capsid (VP1 protein) |
| 3 | AAV2-RC02 capsid (VP1 protein) |
| 4 | AAV2-RC03 capsid (VP1 protein) |
| 5 | AAV2-RC04 capsid (VP1 protein) |
| 6 | AAV2-RC05 capsid (VP1 protein) |
| 7 | AAV2-RC06 capsid (VP1 protein) |
| 8 | AAV2-RC07 capsid (VP1 protein) |
| 9 | AAV2-RC08 capsid (VP1 protein) |
| 10 | AAV2-FT01 capsid (VP1 protein) |
| 11 | AAV2-FT02 capsid (VP1 protein) |
| 12 | AAV2-FT03 capsid (VP1 protein) |
| 13 | AAV2-FT04 capsid (VP1 protein) |
| 14 | AAV2-FT05 capsid (VP1 protein) |
| 15 | AAV2-FT06 capsid (VP1 protein) |
| 16 | AAV2-FT07 capsid (VP1 protein) |
| 17 | AAV2-FT08 capsid (VP1 protein) |
| 18 | AAV2-FT09 capsid (VP1 protein) |
| 19 | AAV2-FT10 capsid (VP1 protein) |
| 20 | AAV2-FT11 capsid (VP1 protein) |
| 21 | AAV2-FT12 capsid (VP1 protein) |
| 22 | STTHLSPPQ peptide insert |
| 23 | SELEEMNNK peptide insert |
| 24 | SPSSPAPAQ peptide insert |
| 25 | RPETQAKPQ peptide insert |
| 26 | STAYTPAPQ peptide insert |
| 27 | RSQRETVWK peptide insert |
| 28 | SVMMVGGRE peptide insert |
| 29 | RKDPEVSEQ peptide insert |
| 30 | SKTNLDRAQ peptide insert |
| 31 | SATQPYASQ peptide insert |
| 32 | RTSAHQVGE peptide insert |
| 33 | RSPKGSAWE peptide insert |
| 34 | SPQQPPRSQ peptide insert |
| 35 | SSRTPQHKQ peptide insert |
| 36 | SPRHPVTTQ peptide insert |
| 37 | SHTTGSPAK peptide insert |
| 38 | RMTGKTGYE peptide insert |
| 39 | RQATSGFDK peptide insert |
| 40 | RAKEMRSEQ peptide insert |
| 41 | SAFSQSAVK peptide insert |

### Capsid Polypeptides

The present disclosure is predicated in part on the identification of novel AAV capsid polypeptides. The capsid polypeptides, when present in the capsid of an AAV vector, can facilitate transduction of cells in vivo, and in particular transduction of hepatocytes (e.g. human hepatocytes). The in vivo transduction of hepatocytes by AAV vectors having a capsid comprising a capsid polypeptide of the present disclosure is generally increased or enhanced compared to AAV vectors comprising a reference AAV capsid polypeptide (e.g. the prototypic AAV2 capsid set forth in SEQ ID NO:1). Transduction or transduction efficiency of AAV vectors can be increased by at least or about 10%, 20%, 30%, 40%, 50%, 60%, 70%, 80%, 90%, 100%, 200%, 300%, 400%, 500%, 600%, 700%, 800%, 900%, 1000% or more, e.g. an AAV vector comprising a capsid polypeptide of the present disclosure can be at least or about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to an AAV vector comprising a reference AAV capsid polypeptide (e.g. one set forth in SEQ ID NO:1). In particular examples, the increased transduction or transduction efficiency of the AAV vector is observed in human hepatocytes in vivo.

The capsid polypeptides of the present disclosure are therefore particularly useful in preparing AAV vectors, and in particular AAV vectors for delivery of heterologous nucleic acid to the liver, such as for therapy of a liver-associated disease or condition. In exemplary embodiments, the capsid polypeptides of the present disclosure are useful in preparing AAV vectors that transduce human hepatocytes in vivo.

The AAV capsid polypeptides of the present disclosure (including isolated capsid polypeptides) include those having a peptide modification in variable region 8 (VRVIII) relative to a reference AAV capsid polypeptide, such as the prototypic AAV2 capsid polypeptide set forth in SEQ ID NO:1 (where VRVIII spans amino acids 579-594 of SEQ ID NO:1). The peptide modification comprises a 7 amino acid insertion relative to the AAV2 capsid polypeptide set forth in SEQ ID NO:1, wherein the 7 amino acid insertion comprises a sequence set forth at amino acids 2-8 of any one of SEQ ID NOs: 22-41. Typically, the peptide modification comprises 9 consecutive amino acid residues having a sequence set forth in any one of SEQ ID NOs: 22-41, which includes the 7 amino acid insertion. The peptide modification can be at any location in VRVIII. In one example, the peptide modification is in the region spanning positions 585-589, with numbering relative to SEQ ID NO:1. In one example, the peptide modification comprises the 7 amino acid insertion after the amino acid residue at positions 587 relative to SEQ ID NO:1, and optionally amino acid substitutions at positions 587 and 588 relative to the AAV2 capsid polypeptide set forth in SEQ ID NO:1, e.g. substitution of the amino acid at position 587 with an arginine or serine, and/or substitution of the amino acid at position 588 with an glutamic acid, glutamine or lysine. In further embodiments, there is, or is also, one or more amino acid substitutions at position 585, 586, and/or 589 relative to SEQ ID NO:1, such as R585G, G586Q and/or Q589A relative to SEQ ID NO:1. The capsid polypeptides of the present disclosure can include all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1). The capsid polypeptides typically comprise at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the VP1, VP2 or VP3 proteins of the prototypic AAV2 set forth in SEQ ID NO:1. In particular embodiments, the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to the VP1, VP2 or VP3 proteins of the prototypic AAV2 set forth in SEQ ID NO:1.

Thus, provided herein are polypeptides, including isolated polypeptides, comprising all or a portion of an AAV capsid polypeptide set forth in any one of SEQ ID NOs: 2-21, including all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1), and variants thereof, including variants comprising at least or about 85%, 86%, 87%, 88%, 89%, 90%, 91%, 92%, 93%, 94%, 95%, 96%, 97%, 98%, or 99% sequence identity to the VP1, VP2 or VP3 proteins described herein. Capsid polypeptides of the disclosure therefore include those comprising all or a portion of the VP1 protein set forth in any one of SEQ ID NOs:2-21, or a polypeptide comprising at least or about 85%, 86%, 87%, 88%, 89%, 90%, 91%, 92%, 93%, 94%, 95%, 96%, 97%, 98%, or 99% sequence identity thereto, wherein the polypeptide comprises a 9 amino acid sequence set forth in any one of SEQ ID NOs: 22-41 at positions 587-595 relative to SEQ ID NOs:2-21, or a 7 amino acid sequence set forth as amino acids 2-8 of any one of SEQ ID NOs: 22-41 at positions 588-594 relative to SEQ ID NOs:2-21. Also included in the present disclosure are capsid polypeptides comprising all or a portion of the VP2 protein set forth as amino acids 138-742 of any one of SEQ ID NOs:2-21 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-742 of SEQ ID NOs:2-21 or a functional fragment thereof, wherein the polypeptide comprises a 9 amino acid sequence set forth in any one of SEQ ID NOs: 22-41 at positions 587-595 relative to SEQ ID NOs:2-21, or a 7 amino acid sequence set forth as amino acids 2-8 of any one of SEQ ID NOs: 22-41 at positions 588-594 relative to SEQ ID NOs:2-21. In addition, provided are capsid polypeptides comprising all or a portion of the VP3 protein set forth as amino acids 203-742 of any one of SEQ ID NOs:2-21 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-742 of any one of SEQ ID NOs:2-21 or a functional fragment thereof, wherein the polypeptide comprises a 9 amino acid sequence set forth in any one of SEQ ID NOs: 22-41 at positions 587-595 relative to SEQ ID NOs:2-21, or a 7 amino acid sequence set forth as amino acids 2-8 of any one of SEQ ID NOs: 22-41 at positions 588-594 relative to SEQ ID NOs:2-21.

An exemplary capsid polypeptide, AAV2-RC01 (SEQ ID NO:2) comprises a peptide modification in VRVIII relative to SEQ ID NO:1, wherein the peptide modification comprises the sequence STTHLSPPQ (SEQ ID NO:22), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence STTHLSPPQ (SEQ ID NO:22), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:2, the VP3 protein set forth as amino acids 203-742 of SEQ ID NO:2, or the VP2 protein set forth as amino acids 138-742 of SEQ ID NO:2 (i.e. the capsid polypeptide comprises a) the sequence of the VP1 protein set forth in SEQ ID NO:2, the VP3 protein set forth as amino acids 203-742 of SEQ ID NO:2, or the VP2 protein set forth as amino acids 138-742 of SEQ ID NO:2; orb) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence STTHLSPPQ (SEQ ID NO:22) at positions 587-595 or the sequence set forth at amino acid positions 2-8 of SEQ ID NO:22 at positions 588-594, with numbering relative to SEQ ID NO:2). In particular embodiments, the portion of the capsid polypeptide that is not the peptide modification (e.g. that is not the 9 amino acid residues set forth in SEQ ID NO:22 at positions 587-595, with numbering relative to SEQ ID NO:2) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-RC02 (SEQ ID NO:3) comprises a peptide modification in VRVIII relative to SEQ ID NO:1, wherein the peptide modification comprises the sequence SELEEMNNK (SEQ ID NO:23), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SELEEMNNK (SEQ ID NO:23), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:3, the VP3 protein set forth as amino acids 203-742 of SEQ ID NO:3, or the VP2 protein set forth as amino acids 138-742 of SEQ ID NO:3 (i.e. the capsid polypeptide comprises a) the sequence of the VP1 protein set forth in SEQ ID NO:3, the VP3 protein set forth as amino acids 203-742 of SEQ ID NO:3, or the VP2 protein set forth as amino acids 138-742 of SEQ ID NO:3; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SELEEMNNK (SEQ ID NO:23) at positions 587-595 or the sequence set forth at amino acid positions 2-8 of SEQ ID NO:23 at positions 588-594, with numbering relative to SEQ ID NO:3). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-RC03 (SEQ ID NO:4) comprises a peptide modification in VRVIII relative to SEQ ID NO:1, wherein the peptide modification comprises the sequence SPSSPAPAQ (SEQ ID NO:24), which includes a 7 amino acid insertion after position 587 relative to the prototypic AAV2 capsid polypeptide set forth in SEQ ID NO:1, and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SPSSPAPAQ (SEQ ID NO:24), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:4, the VP3 protein set forth as amino acids 203-742 of SEQ ID NO:4, or the VP2 protein set forth as amino acids 138-742 of SEQ ID NO:4 (i.e. the capsid polypeptide comprises a) the sequence of the VP1 protein set forth in SEQ ID NO:4, the VP3 protein set forth as amino acids 203-742 of SEQ ID NO:4, or the VP2 protein set forth as amino acids 138-742 of SEQ ID NO:4; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SPSSPAPAQ (SEQ ID NO:24) at positions 587-595 or the sequence set forth at amino acid positions 2-8 of SEQ ID NO:24 at positions 588-594, with numbering relative to SEQ ID NO:4). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-RC04 (SEQ ID NO:5) comprises a peptide modification in VRVIII relative to SEQ ID NO:1, wherein the peptide modification comprises the sequence RPETQAKPQ (SEQ ID NO:25), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RPETQAKPQ (SEQ ID NO:25), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:5, the VP3 protein set forth as amino acids 203-742 of SEQ ID NO:5, or the VP2 protein set forth as amino acids 138-742 of SEQ ID NO:5 (i.e. the capsid polypeptide comprises a) the sequence of the VP1 protein set forth in SEQ ID NO:5, the VP3 protein set forth as amino acids 203-742 of SEQ ID NO:5, or the VP2 protein set forth as amino acids 138-742 of SEQ ID NO:5; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RPETQAKPQ (SEQ ID NO:25) at positions 587-595 or the sequence set forth at amino acid positions 2-8 of SEQ ID NO:25 at positions 588-594, with numbering relative to SEQ ID NO:5). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-RC05 (SEQ ID NO:6) comprises a peptide modification in VRVIII relative to SEQ ID NO:1, wherein the peptide modification comprises the sequence STAYTPAPQ (SEQ ID NO:26), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence STAYTPAPQ (SEQ ID NO:26), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:6, the VP3 protein set forth as amino acids 203-742 of SEQ ID NO:6, or the VP2 protein set forth as amino acids 138-742 of SEQ ID NO:6 (i.e. the capsid polypeptide comprises a) the sequence of the VP1 protein set forth in SEQ ID NO:6, the VP3 protein set forth as amino acids 203-742 of SEQ ID NO:6, or the VP2 protein set forth as amino acids 138-742 of SEQ ID NO:6; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence STAYTPAPQ (SEQ ID NO:26) at positions 587-595 or the sequence set forth at amino acid positions 2-8 of SEQ ID NO:26 at positions 588-594, with numbering relative to SEQ ID NO:6). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide AAV2-RC06 (SEQ ID NO:7) comprises a peptide modification in VRVIII relative to SEQ ID NO:1, wherein the peptide modification comprises the sequence RSQRETVWK (SEQ ID NO:27), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RSQRETVWK (SEQ ID NO:27), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:7, the VP3 protein set forth as amino acids 203-742 of SEQ ID NO:7, or the VP2 protein set forth as amino acids 138-742 of SEQ ID NO:7 (i.e. the capsid polypeptide comprises a) the sequence of the VP1 protein set forth in SEQ ID NO:7, the VP3 protein set forth as amino acids 203-742 of SEQ ID NO:7, or the VP2 protein set forth as amino acids 138-742 of SEQ ID NO:7; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RSQRETVWK (SEQ ID NO:27) at positions 587-595 or the sequence set forth at amino acid positions 2-8 of SEQ ID NO:27 at positions 588-594, with numbering relative to SEQ ID NO:7). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-RC07 (SEQ ID NO:8) comprises a peptide modification in VRVIII relative to SEQ ID NO:1, wherein the peptide modification comprises the sequence SVMMVGGRE (SEQ ID NO:28), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SVMMVGGRE (SEQ ID NO:28), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:8, the VP3 protein set forth as amino acids 203-742 of SEQ ID NO:8, or the VP2 protein set forth as amino acids 138-742 of SEQ ID NO:8 (i.e. the capsid polypeptide comprises a) the sequence of the VP1 protein set forth in SEQ ID NO:8, the VP3 protein set forth as amino acids 203-742 of SEQ ID NO:8, or the VP2 protein set forth as amino acids 138-742 of SEQ ID NO:8; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SVMMVGGRE (SEQ ID NO:28) at positions 587-595 or the sequence set forth at amino acid positions 2-8 of SEQ ID NO:28 at positions 588-594, with numbering relative to SEQ ID NO:8). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-RC08 (SEQ ID NO:9) comprises a peptide modification in VRVIII relative to SEQ ID NO:1, wherein the peptide modification comprises the sequence RKDPEVSEQ (SEQ ID NO:29), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RKDPEVSEQ (SEQ ID NO:29), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:9, the VP3 protein set forth as amino acids 203-742 of SEQ ID NO:9, or the VP2 protein set forth as amino acids 138-742 of SEQ ID NO:9 (i.e. the capsid polypeptide comprises a) the sequence of the VP1 protein set forth in SEQ ID NO:9, the VP3 protein set forth as amino acids 203-742 of SEQ ID NO:9, or the VP2 protein set forth as amino acids 138-742 of SEQ ID NO:9; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RKDPEVSEQ (SEQ ID NO:29) at positions 587-595 or the sequence set forth at amino acid positions 2-8 of SEQ ID NO:29 at positions 588-594, with numbering relative to SEQ ID NO:9). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-FT01 (SEQ ID NO:10) comprises a peptide modification in VRVIII relative to SEQ ID NO:1, wherein the peptide modification comprises the sequence SKTNLDRAQ (SEQ ID NO:30), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SKTNLDRAQ (SEQ ID NO:30), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:10, the VP3 protein set forth as amino acids 203-742 of SEQ ID NO:10, or the VP2 protein set forth as amino acids 138-742 of SEQ ID NO:10 (i.e. the capsid polypeptide comprises a) the sequence of the VP1 protein set forth in SEQ ID NO:10, the VP3 protein set forth as amino acids 203-742 of SEQ ID NO:10, or the VP2 protein set forth as amino acids 138-742 of SEQ ID NO:10; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SKTNLDRAQ (SEQ ID NO:30) at positions 587-595 or the sequence set forth at amino acid positions 2-8 of SEQ ID NO:30 at positions 588-594, with numbering relative to SEQ ID NO:10). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-FT02 (SEQ ID NO:11) comprises a peptide modification in VRVIII relative to SEQ ID NO:1, wherein the peptide modification comprises the sequence SATQPYASQ (SEQ ID NO:31), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SATQPYASQ (SEQ ID NO:31), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:11, the VP3 protein set forth as amino acids 203-742 of SEQ ID NO:11, or the VP2 protein set forth as amino acids 138-742 of SEQ ID NO:11 (i.e. the capsid polypeptide comprises a) the sequence of the VP1 protein set forth in SEQ ID NO:11, the VP3 protein set forth as amino acids 203-742 of SEQ ID NO:11, or the VP2 protein set forth as amino acids 138-742 of SEQ ID NO:11; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SATQPYASQ (SEQ ID NO:31) at positions 587-595 or the sequence set forth at amino acid positions 2-8 of SEQ ID NO:31 at positions 588-594, with numbering relative to SEQ ID NO:11). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-FT03 (SEQ ID NO:12) comprises a peptide modification in VRVIII relative to SEQ ID NO:1, wherein the peptide modification comprises the sequence RTSAHQVGE (SEQ ID NO:32), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RTSAHQVGE (SEQ ID NO:32), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:12, the VP3 protein set forth as amino acids 203-742 of SEQ ID NO:12, or the VP2 protein set forth as amino acids 138-742 of SEQ ID NO:12 (i.e. the capsid polypeptide comprises a) the sequence of the VP1 protein set forth in SEQ ID NO:12, the VP3 protein set forth as amino acids 203-742 of SEQ ID NO:12, or the VP2 protein set forth as amino acids 138-742 of SEQ ID NO:12; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RTSAHQVGE (SEQ ID NO:32) at positions 587-595 or the sequence set forth at amino acid positions 2-8 of SEQ ID NO:32 at positions 588-594, with numbering relative to SEQ ID NO:12). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-FT04 (SEQ ID NO:13) comprises a peptide modification in VRVIII relative to SEQ ID NO:1, wherein the peptide modification comprises the sequence RSPKGSAWE (SEQ ID NO:33), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RSPKGSAWE (SEQ ID NO:33), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:13, the VP3 protein set forth as amino acids 203-742 of SEQ ID NO:13, or the VP2 protein set forth as amino acids 138-742 of SEQ ID NO:13 (i.e. the capsid polypeptide comprises a) the sequence of the VP1 protein set forth in SEQ ID NO:13, the VP3 protein set forth as amino acids 203-742 of SEQ ID NO:13, or the VP2 protein set forth as amino acids 138-742 of SEQ ID NO:13; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RSPKGSAWE (SEQ ID NO:33) at positions 587-595 or the sequence set forth at amino acid positions 2-8 of SEQ ID NO:33 at positions 588-594, with numbering relative to SEQ ID NO:13). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-FT05 (SEQ ID NO:14) comprises a peptide modification in VRVIII relative to SEQ ID NO:1, wherein the peptide modification comprises the sequence SPQQPPRSQ (SEQ ID NO:34), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SPQQPPRSQ (SEQ ID NO:34), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:14, the VP3 protein set forth as amino acids 203-742 of SEQ ID NO:14, or the VP2 protein set forth as amino acids 138-742 of SEQ ID NO:14 (i.e. the capsid polypeptide comprises a) the sequence of the VP1 protein set forth in SEQ ID NO:14, the VP3 protein set forth as amino acids 203-742 of SEQ ID NO:14, or the VP2 protein set forth as amino acids 138-742 of SEQ ID NO:14; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SPQQPPRSQ (SEQ ID NO:34) at positions 587-595 or the sequence set forth at amino acid positions 2-8 of SEQ ID NO:34 at positions 588-594, with numbering relative to SEQ ID NO:14). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide AAV2-FT06 (SEQ ID NO:15) comprises a peptide modification in VRVIII relative to SEQ ID NO:1, wherein the peptide modification comprises the sequence SSRTPQHKQ (SEQ ID NO:35), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SSRTPQHKQ (SEQ ID NO:35), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:15, the VP3 protein set forth as amino acids 203-742 of SEQ ID NO:15, or the VP2 protein set forth as amino acids 138-742 of SEQ ID NO:15 (i.e. the capsid polypeptide comprises a) the sequence of the VP1 protein set forth in SEQ ID NO:15, the VP3 protein set forth as amino acids 203-742 of SEQ ID NO:15, or the VP2 protein set forth as amino acids 138-742 of SEQ ID NO:15; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SSRTPQHKQ (SEQ ID NO:35) at positions 587-595 or the sequence set forth at amino acid positions 2-8 of SEQ ID NO:35 at positions 588-594, with numbering relative to SEQ ID NO:15). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide AAV2-FT07 (SEQ ID NO:16) comprises a peptide modification in VRVIII relative to SEQ ID NO:1, wherein the peptide modification comprises the sequence SPRHPVTTQ (SEQ ID NO:36), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SPRHPVTTQ (SEQ ID NO:36), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:16, the VP3 protein set forth as amino acids 203-742 of SEQ ID NO:16, or the VP2 protein set forth as amino acids 138-742 of SEQ ID NO:16 (i.e. the capsid polypeptide comprises a) the sequence of the VP1 protein set forth in SEQ ID NO:16, the VP3 protein set forth as amino acids 203-742 of SEQ ID NO:16, or the VP2 protein set forth as amino acids 138-742 of SEQ ID NO:16; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SPRHPVTTQ (SEQ ID NO:36) at positions 587-595 or the sequence set forth at amino acid positions 2-8 of SEQ ID NO:36 at positions 588-594, with numbering relative to SEQ ID NO:16). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-FT08 (SEQ ID NO:17) comprises a peptide modification in VRVIII relative to SEQ ID NO:1, wherein the peptide modification comprises the sequence SHTTGSPAK (SEQ ID NO:37), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SHTTGSPAK (SEQ ID NO:37), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:17, the VP3 protein set forth as amino acids 203-742 of SEQ ID NO:17, or the VP2 protein set forth as amino acids 138-742 of SEQ ID NO:17 (i.e. the capsid polypeptide comprises a) the sequence of the VP1 protein set forth in SEQ ID NO:17, the VP3 protein set forth as amino acids 203-742 of SEQ ID NO:17, or the VP2 protein set forth as amino acids 138-742 of SEQ ID NO:17; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SHTTGSPAK (SEQ ID NO:37) at positions 587-595 or the sequence set forth at amino acid positions 2-8 of SEQ ID NO:37 at positions 588-594, with numbering relative to SEQ ID NO:17). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-FT09 (SEQ ID NO:18) comprises a peptide modification in VRVIII relative to SEQ ID NO:1, wherein the peptide modification comprises the sequence RMTGKTGYE (SEQ ID NO:38), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RMTGKTGYE (SEQ ID NO:38), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:18, the VP3 protein set forth as amino acids 203-742 of SEQ ID NO:18, or the VP2 protein set forth as amino acids 138-742 of SEQ ID NO:18 (i.e. the capsid polypeptide comprises a) the sequence of the VP1 protein set forth in SEQ ID NO:18, the VP3 protein set forth as amino acids 203-742 of SEQ ID NO:18, or the VP2 protein set forth as amino acids 138-742 of SEQ ID NO:18; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RMTGKTGYE (SEQ ID NO:38) at positions 587-595 or the sequence set forth at amino acid positions 2-8 of SEQ ID NO:38 at positions 588-594, with numbering relative to SEQ ID NO:18). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-FT10 (SEQ ID NO:19) comprises a peptide modification in VRVIII relative to SEQ ID NO:1, wherein the peptide modification comprises the sequence RQATSGFDK (SEQ ID NO:39), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RQATSGFDK (SEQ ID NO:39), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:19, the VP3 protein set forth as amino acids 203-742 of SEQ ID NO:19, or the VP2 protein set forth as amino acids 138-742 of SEQ ID NO:19 (i.e. the capsid polypeptide comprises a) the sequence of the VP1 protein set forth in SEQ ID NO:19, the VP3 protein set forth as amino acids 203-742 of SEQ ID NO:19, or the VP2 protein set forth as amino acids 138-742 of SEQ ID NO:19; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RQATSGFDK (SEQ ID NO:39) at positions 587-595 or the sequence set forth at amino acid positions 2-8 of SEQ ID NO:39 at positions 588-594, with numbering relative to SEQ ID NO:19). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-FT11 (SEQ ID NO:20) comprises a peptide modification in VRVIII relative to SEQ ID NO:1, wherein the peptide modification comprises the sequence RAKEMRSEQ (SEQ ID NO:40), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RAKEMRSEQ (SEQ ID NO:40), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:20, the VP3 protein set forth as amino acids 203-742 of SEQ ID NO:20, or the VP2 protein set forth as amino acids 138-742 of SEQ ID NO:20 (i.e. the capsid polypeptide comprises a) the sequence of the VP1 protein set forth in SEQ ID NO:20, the VP3 protein set forth as amino acids 203-742 of SEQ ID NO:20, or the VP2 protein set forth as amino acids 138-742 of SEQ ID NO:20; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RAKEMRSEQ (SEQ ID NO:40) at positions 587-595 or the sequence set forth at amino acid positions 2-8 of SEQ ID NO:40 at positions 588-594, with numbering relative to SEQ ID NO:20). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-FT12 (SEQ ID NO:21) comprises a peptide modification in VRVIII relative to SEQ ID NO:1, wherein the peptide modification comprises the sequence SAFSQSAVK (SEQ ID NO:41), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SAFSQSAVK (SEQ ID NO:41), and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:21, the VP3 protein set forth as amino acids 203-742 of SEQ ID NO:21, or the VP2 protein set forth as amino acids 138-742 of SEQ ID NO:21 (i.e. the capsid polypeptide comprises a) the sequence of the VP1 protein set forth in SEQ ID NO:21, the VP3 protein set forth as amino acids 203-742 of SEQ ID NO:21, or the VP2 protein set forth as amino acids 138-742 of SEQ ID NO:21; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SAFSQSAVK (SEQ ID NO:41) at positions 587-595 or the sequence set forth at amino acid positions 2-8 of SEQ ID NO:41 at positions 588-594, with numbering relative to SEQ ID NO:4). In particular embodiments, the portion of the capsid polypeptide that is not the peptide modification has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Also provided are nucleic acid molecules, including isolated nucleic acid molecules, encoding a capsid polypeptide of the disclosure. Thus, amongst the nucleic acid molecules provided herein are those encoding a capsid polypeptide comprising the VP1, VP2 and/or VP3 of any one of the capsid polypeptides set forth in SEQ ID NOs:2-21 as described above or a polypeptide having at least or about 85%, 86%, 87%, 88%, 89%, 90%, 91%, 92%, 93%, 94%, 95%, 96%, 97%, 98%, or 99% sequence identity thereto.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein. Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, HeLa cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instance, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject, and in particular the liver of a subject, treats a liver-associated disease or condition (i.e. a disease or condition with a pathology that manifests at least in part in the liver, and/or is caused at least in part by expression of one or more genes in the liver). In illustrative embodiments, the liver-associated disease or condition is selected from among a urea cycle disorder (UCD; including N-acetylglutamate synthase deficiency (NAGSD), carbamylphosphate synthetase 1 deficiency (CPS1D), ornithine transcarbamylase deficiency (OTCD), argininosuccinate synthetase deficiency (ASSD), argininosuccinate lyase (ASLD), arginase 1 deficiency (ARG1D), citrin or aspartate/glutamate carrier deficiency and the mitochondrial ornithine transporter 1 deficiency causing hyperornithinemia-hyperammonemia-homocitrullinuria syndrome (HHH syndrome)), organic acidopathy (or organic academia, including methylmalonic acidemia, propionic acidemia, isovaleric acidemia, and maple syrup urine disease), aminoacidopathy, glycogenoses (Types I, III and IV), Wilson's disease, Progressive Familial Intrahepatic Cholestasis, primary hyperoxaluria, complementopathy, coagulopathy (e.g. hemophilia A, hemophilia B, von Willebrand disease (VWD)), Crigler Najjar syndrome, familial hypercholesterolaemia, α-1-antitrypsin deficiency, mitochondria respiratory chain hepatopathy, and citrin deficiency. Those skilled in the art would readily be able to select an appropriate heterologous coding sequence useful for treating such diseases. In some examples, the heterologous coding sequence comprises all or a part of a gene that is associated with the disease, such as all or a part of a gene set forth in Table 2. Introduction of such a sequence to the liver can be used for gene replacement or gene editing/correction, e.g. using CRISPR-Cas9. In particular examples, the heterologous coding sequence encodes a protein encoded by a gene that is associated with the disease, such as a gene set forth in Table 2.

TABLE 2

| Exemplary liver-associated diseases | Exemplary associated genes |
|---|---|
| Urea cycle disorders (UCDs) | OTC, ASS, CPS1, ASL, ARG1 |
| Organic acidopathies | PCCA, PCCB, MMUT |
| Aminoacidopathies | PAH, FAH |
| Glycogenoses (Types I, III and IV) | SLC37A4 |
| Wilson's Disease | ATP7B |
| Progressive Familial Intrahepatic | ABCB4, ABCB11, ATP8B1 |
| Cholestasis | |
| Primary Hyperoxaluria | AGXT |

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCI or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

The vectors of the present disclosure can comprise promoters. In instances where the vector is a nucleic acid vector comprising nucleic acid encoding the capsid polypeptide, the promoter may facilitate expression of the nucleic acid encoding the capsid polypeptide. In instances where the vector is an AAV vector, the promoter may facilitate expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. Non-limiting examples of such promoters include the liver-specific thyroxin binding globulin (TBG) promoter, insulin promoter, glucagon promoter, somatostatin promoter, pancreatic polypeptide (PPY) promoter, synapsin-1 (Syn) promoter, creatine kinase (MCK) promoter, mammalian desmin (DES) promoter, a α-myosin heavy chain (a-MHC) promoter, a cardiac Troponin T (cTnT) promoter, beta-actin promoter, and hepatitis B virus core promoter. The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a nucleic acid molecule or vector or of the present disclosure. In some instances, the host cells are used to amplify, replicate, package and/or purify a polynucleotide or vector. In other examples, the host cells are used to express a heterologous sequence, such as one packaged within AAV vector. Exemplary host cells include prokaryotic and eukaryotic cells. In some instances, the host cell is a mammalian host cell. It is well within the skill of a skilled artisan to select an appropriate host cell for the expression, amplification, replication, packaging and/or purification of a polynucleotide, vector or rAAV virion of the present disclosure. Exemplary mammalian host cells include, but are not limited to, HEK293 cells, HeLa cells, Vero cells, HuH-7 cells, and HepG2 cells. In particular examples, the host cell is a hepatocyte or cell-line derived from a hepatocyte.

### Compositions and Methods

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TweenTm, PluronicsTm or polyethylene glycol (PEG). In some embodiments, the physiologically acceptable carrier is an aqueous pH buffered solution.

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a hepatocyte, such as a human hepatocyte.

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titers of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intravenous, intraperitoneal, subcutaneous, epicutaneous, intradermal, intramuscular, pulmonary, intracranial, intraosseous, oral, buccal, or nasal routes. The AAV vector can be administered as a single dose or multiple doses, and at varying intervals.

Also provided are methods for producing an AAV vector described above and herein, i.e. one comprising a capsid polypeptide of the present disclosure. Such methods comprise culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Materials and Methods

### Cell Culture Conditions and Cell Origins

AAV production was performed in a human embryonic kidney (HEK) 293T cell line (ATCC, Cat# CRL-3216) and grown in Dulbecco's modified Eagle's medium (DMEM, Gibco, Cat# 11965) supplemented with 10% fetal bovine serum (FBS, Sigma-Aldrich, Cat# F9423), 1× Pen Strep (Gibco, Cat# 15070), and 25 mM HEPES (Gibco, Cat# 15630).

### PCR Reactions

Standard and Illumina amplicon-seq NGS polymerase chain reactions (PCRs) were performed using Q5 [NEB, Cat# M0491], dNTPs [NEB, Cat# N0447] and primers (all Sigma-Aldrich) described below using the following standard protocol: initial denaturation: 98° C. for 10 sec, denaturation: 98° C. for 10 sec, annealing: unique for each oligo based on https://tmcalculator.neb.com for 10 sec, extension: 72° C. for 20 sec per 1000 nt, final extension: 72° C. for 10 min. The PCR products were run on agarose gels (Bioline, Cat# BIO-41025), using 1% agarose/1× Tris-acetate-EDTA (TAE, Invitrogen, Cat#24710-030) for products over 500 bp, and 2% agarose/TAE for products smaller than 500 bp. The DNA was extracted using the Zymoclean Gel DNA Recovery Kit (Zymogen, Cat# D4001) following the manufacturer's instructions.

### Plasmid Preparations

Novel capsid variants selected in the peptide library screen as well as the capsid FT11 were cloned using two primers binding to the pRep2Caplco2_SfiI plasmid and each bearing half of the peptide-coding region as a long overhang. The whole plasmid was amplified using the respective primers, reassembled using KLD enzyme mix (NEB, Cat# M0554), and transformed into bacteria. The remaining FT-selected capsids (FT01-10 & 12, n=11) were cloned using a universal reverse primer for all peptides and individual forward primers with very long 5′-extensions bearing the entire peptide sequence.

### Library Preparation

For the preparation of the AAV2 peptide display library, double SfiI restriction sites were inserted into the local codon-optimized version of the AAV2 cap gene (Caplco2) at the N587 insertion site, as described previously (Logan et al., 2017, Nat Genet 49: 1267). In brief, using primers with very long overhangs and 18 nt homology arms at the 3′end between each other, the pRep2Caplco2 plasmid was amplified and the added regions (primer overhangs bearing SfiI sites) were incorporated using NEBuilder (NEB, Cat# E2621). The resulting plasmid pRep2Caplco2_SfiI was then digested using SwaI and NsiI, and the capsid-containing fragment was ligated into the equally SwaI/NsiI digested FT-SFFV selection platform. This FT-SFFV-Ico2_SfiI construct was now digested with SfiI overnight, purified using the QlAquick PCR Purification Kit (QIAGEN, Cat# 28104), and re-digested with SfiI overnight. Lastly, the construct was dephosphorylated using calf intestinal alkaline phosphatase (NEB, Cat# M0290). Following electrophoretic separation and gel extraction (Zymogen, Cat# D4001), the backbone was ready for accepting the peptide library-coding fragment.

The peptide library itself was ordered as an oligo nucleotide with 20 nt homologies to each end of the SfiI-digested FT-SFFV-Ico2_SfiI backbone flanking a NNK7 motif coding for randomized amino acids with lower redundancy (Muller et al. 2003, Nat Biotechnol 21(9): 1040-1046). In addition to the seven random amino acids, the ‘SfiI-clipped’ codons upstream (arginine or serine) and downstream (glutamine, lysine or glutamate) of the random insertion were coded to be semi-variable (full oligo as ordered in reverse complement, Ico2_NNK7). Therefore, the library contained a 7-mer random insert flanked by two variable amino acids coding for 9-mer novel peptides. Before insertion into the backbone, the oligonucleotide was made double stranded using a short primer binding on the homology arm upstream of the peptide (lco2-dsSyn), Klenow (exo-) (NEB, Cat# M0212), and dNTPs (NEB, Cat# N0447). The fragment (dslco2-library) was gel purified and ready for insertion.

The final library was generated by mixing 225 fmol of the digested FT-SFFV-Ico2_SfiI backbone with 2250 fmol of the dslco2-library insert into 13 individual NEBuilder (NEB, Cat# E2621) reactions. The reactions were combined after assembly and purified using ethanol precipitation. The resulting pellet (1 μg of DNA) was used for electroporation into competent cells (Lucigen, Cat# 60512). The recovered transformants were used to inoculate 250 mL lysogeny broth (LB) containing 10 μg/mL trimethoprim (TMP). Only 10 μL of recovered transformants were used to plate a 10⁻¹-10⁻⁵ dilution series on TMP-LB-agar plates to determine transformation efficiency (2.3×10⁷ colonies per μg of DNA). The 250 mL of inculcated LB were maxi prepped (Invitrogen, Cat# A31217).

To move the same library into the RC platform, the pFT-SFFV-Ico2_7mer was digested alongside the pRC platform using SawI/NsiI. One pg of the RC backbone was ligated with the digested Ico2_7mer capsid at a 1:3 ratio and electroporated as described above.

### AAV Production

All production types required pAd5 helper plasmids and, in some cases, other helpers as indicated throughout the section. The 20 novel AAV2 peptide display variants as well as benchmarks AAV2 and AAV NP59 for validating novel capsids in primary human hepatocytes in vivo were produced in 5×15 cm dishes of HEK293T cells to package three unique single-stranded ITR2-LSP-eGFP-N₆Barcode(BC)-WPRE-ITR2 transgenes each as described previously (Cabanes-Creus et al., 2020, Science Translational Medicine 12(560): eaba3312; Cabanes-Creus et al., 2020, Mol Ther Methods Clin Dev 17: 1139-1154). The RC-Ico2_7mer and FT-SFFV-Ico2_7mer libraries (alongside pRep2 helper plasmids) were produced in 5×15 cm dishes of HEK293T cells. Five μg of transgene plasmid per dish were transfected for the LSP-GFP construct and 250 ng (˜500 copies per cell) of transgene plasmid per dish were transfected for library production to reduce cross-packaging. All aforementioned constructs were harvested and purified using iodixanol ultra-centrifugation, as described previously (Cabanes-Creus et al., 2020, Mol Ther Methods Clin Dev 17: 1139-1154).

Iodixanol-produced AAV were quantified using droplet digital PCR (ddPCR, (Bio-Rad, Berkeley) using QX200 ddPCR EvaGreen Supermix (Cat# 1864034; Bio-Rad) with eGFP primers, with the exception of the RC-Ico2_7mer libraries, which were quantified using Rep2 primers, as described previously (Cabanes-Creus et al., 2019, Mol Ther Methods Clin Dev 12: 71-84).

The resulting AAVs were titrated using real-time quantitative polymerase chain reaction (qPCR) master mix (Cat# 172-5125; Bio-Rad) with serial dilutions of a linearized plasmid as a standard curve and eGFP_F/R and Rep2_F/R primers (Cabanes-Creus et al., 2019, Mol Ther Methods Clin Dev 12: 71-84). In addition to Benzonase treatment as part of the crude lysate preparation, the clarified lysates were treated with DNAseI (NEB , Cat# M0303) to improve removal of any remaining plasmid or genomic DNA prior to Proteinase K (NEB, Cat# P8107) digest and subsequent qPCR reaction.

Animal Work

Mouse studies were supported by the Bio Resources Core Facility at the Children's Medical Research Institute (CMRI). All animal care and experimental procedures were approved by the joint CMRI and The Children's Hospital at Westmead Animal Care and Ethics Committee. The Fah^{-/-}Rag2^{-/-}Il2rg^{-/-} (FRG) mice were housed inside a BSL2 facility in individually ventilated cages with 10% 2-(2-nitro-4-trifluoro-methylbenzoyl)-1,3-cyclohexanedione (NTBC)-supplemented in drinking water (Azuma et al., 2007, Nat Biotechnol 25(8): 903-910). Six to eight week old FRG females were engrafted with primary human hepatocytes (Lonza Group Ltd., Basel, Switzerland) as described previously, followed by on/off NTBC cycles to promote the expansion of human cells (Azuma et al., 2007, Nat Biotechnol 25(8): 903-910; Logan et al. 2017, Nat Genet 49: 1267). Levels of human hepatocyte engraftment in the chimeric liver of humanized FRG (hFRG) mice were assessed by measuring the levels of human albumin in peripheral blood using a Human Albumin ELISA Quantitation Kit (Bethyl, Cat# E80-129), as previously described (Azuma et al., 2007, Nat Biotechnol 25(8): 903-910). Mice were euthanized by isofluorane inhalation one week after transduction for organ and hepatocyte harvests. For fluorescence-activated cell sorting (FACS), hepatocytes were harvested by collagenase perfusion as previously described (Logan et al. 2017, Nat Genet 49: 1267). Single-cell suspensions were obtained by cannulation of the inferior vena cava, and pumped with an osmotic minipump (Gilson Minipuls 3) in the following order: 25 mL of Hank's balanced salt solution (HBSS) (cat# H9394; Sigma), 25 mL of HBSS supplemented with 0.5 mM EDTA, 25 mL HBSS, and 25 mL of HBSS supplemented with 5 mM CaCl₂, 0.05% w/v collagenase IV, and 0.01% w/v DNase I. Following perfusion, the chimeric liver was harvested and placed in a sterile cell culture dish containing DMEM supplemented with 10% FBS. The cells were collected after opening the liver capsule followed by centrifugation at 50×g for 3 min at 4° C. The cell pellet was resuspended in DMEM and passed through a 100 μm nylon cell strainer. Isotonic Percoll (10% 10× PBS and 90% Percoll; GE Healthcare, Cat# 17089102) was added to the cell suspension to separate the live and dead cells. Live cells were pelleted at 650×g for 10 min at 4° C. and the pellet was resuspended in FACS+DAPI buffer. To distinguish between murine and human hepatocytes, cells were labelled with phycoerythrin-conjugated anti-human-HLA-ABC (clone W6/32; Invitrogen Cat# 12-9983-42; 1:20), biotin-conjugated anti-mouse-H2Kb (clone AF6-88.5, BD Pharmigen Cat# 553568; 1:100), and allophycocyanin-conjugated streptavidin (eBioscience Cat# 17-4317-82; 1:500). Labelled samples were sorted to a minimal 95% purity using a BD AriaIII cell sorter. FACS was performed at the Flow Cytometry Facility, Westmead Institute for Medical Research, Westmead, NSW, Australia. The data were analyzed using FlowJo 7.6.1 (FlowJo, LLC).

### AAV2 Peptide Display Library Selections

The in vivo RC-Ico2_7mer library selection in the presence of Ad5 in humanized FRG mice was performed as described previously using the same stock of Ad5 previously validated (Cabanes-Creus et al., 2020, Science Translational Medicine 12(560): eaba3312). In brief, 2×10¹¹ vg of the original RC-lco2_7mer library were injected intravenously into a humanized FRG mouse for the first round of selection, and 250 μL of liver lysate (corresponding to 2×10⁹ vgc for secondary RC library) were injected intra-peritoneally for the second round. Before injection and after each of the two rounds of selection, libraries were analyzed by NGS to track the selection kinetics based on the decreasing number of unique peptides with every round of selection (see below for details).

For the in vivo FT-SFFV-Ico2_7mer library selection, 2×10¹¹ vg were injected into a humanized FRG mouse. One week after injection, the chimeric liver was perfused, and cells were sorted for human HLA and GFP-positivity. DNA and RNA were extracted and processed for cDNA synthesis as well as NGS PCR alongside the original packaged library. In addition, a 180 bp region around the peptide library was amplified from cDNA using primers PepRec_F/R and gel extracted. Then, 2250 fmol of this peptide library-bearing fragment were inserted into 225 fmol of the twice SfiI-digested and de-phosphorylated FT-SFFV-Ico2_SfiI using NEBuilder assembly and electroporated into bacteria, as described in the library generation section above. This secondary library bearing the peptides from expressed capsid genes from round one was packaged and injected into another humanized FRG mice (4×10¹⁰ vg). The perfusion was performed after one week, as explained above, and after the second round, RNA was extracted from human hepatocytes, prepared for cDNA synthesis, and analyzed to track selection kinetics using NGS.

### DNA and RNA Extraction from Cells and Tissue Samples

DNA from the mouse and human hepatocytes from humanized FRG mice were isolated using phenol-chloroform extraction after proteinase K digestion. Briefly, the cells were resuspended in 400 μL lysis buffer (10 mM Tris-HCl pH 8 [Invitrogen, Cat# 15575-020], 0.1 mM EDTA [Invitrogen, Cat# 15575-020], 0.2% (w/v) sodium dodecyl sulphate [Sigma-Aldrich, Cat# 71736-100ML], and RNAse A [Invitrogen, Cat# 12091021]), and incubated at 37° C. for one hour. Afterwards, 50 μg/mL Proteinase K (QIAGEN, Cat# 19131) was added and incubated overnight at 55° C. at 800 rpm rotation in heat block (Thermomix, Eppendorf). The next day, 400 μL of phenol:chloroform:isoamyl alcohol (25:24:1, Sigma-Aldrich, Cat# P3803-100ML) was added and mixed well with the sample. The phases were separated by centrifugation at 21,000×g for 15 min, the top aqueous phase containing DNA was transferred to a new tube and mixed with 0.1× v/v 3 M sodium acetate and 2.5× v/v 100% ice-cold ethanol, incubated for 1 hour at −80° C. and spun at 21,000×g, 4° C. for 20 min. The resulting DNA pellet was washed twice with 75% (v/v) ethanol and resuspended in nuclease-free water.

RNA from the mouse and human hepatocytes from humanized FRG mice were isolated using 1 mL TriReagent (Sigma, Cat# T9424). After cell lysis, 200 μL chloroform (VWR, Cat# 22711.26) was added, mixed, and incubated for 5 min at RT and separated using centrifugation at 21,000×g, at 4° C. for 20 min. We then collected 500 μL aqueous phase and mixed it with 500 μL cold isopropanol (Merck EMD, Cat# 8187661000). The RNA was pelleted at 21,000×g, at 4° C. for 20 min. The RNA pellet was washed twice using ice-cold 75% ethanol and resuspended in nuclease-free water.

### Reverse Transcription of Extracted RNA

750 ng of total RNA was incubated with two units of TURBO DNase (Invitrogen, Cat# AM1907) twice for 1 hour, followed by incubation with DNAse inactivation reagent following the manufacturer's instructions. The DNase-treated RNA was then used for cDNA synthesis using the SuperScript IV First-Strand Synthesis System (Invitrogen, Cat# 18091050) following the manufacturer's instructions using various primers for second-strand synthesis:

2 μM of local codon-optimized AAV capsid 2 reverse primer (lco2_R) for peptide coding region recovery in the FT-RNA library selection in humanized FRG mice.

2 μM of WPRE-binding primer (WPRE_R) to specifically synthesize the barcoded ssAAV-LSP-GFP-BC-WPRE cDNA used to validate novel capsids in humanized FRG mice (LSP).

### Next-Generation Sequencing

The AAV2-based peptide display libraries were NGS-screened at every step of selection, including before selection (packaged library), after round 1 (RC-Ad5, FT-DNA, FT-RNA), and after round 2 (RC-Ad5, FT-RNA), using primers PepLib_F/R.

### Normalization of NGS Reads

NGS data obtained from all samples, except the peptide library screen, were normalized to the barcode contribution of the respective input (as indicated above). Read counts for each sample and each variant were multiplied by the variant specific ‘normalization co-efficient’ of the respective input, which was calculated as follows:

$$Normalization co - efficient = \frac{\% of NGS {reads}^{should}}{\% of NGS {reads}^{measured}}$$

### Selection of Novel Capsids

The novel capsids selected were named after the cell type they were selected in (human hepatocytes, hu.Hep.) and according to the selection platform (RC or FT) as well as the rank in the library contribution after the second round of selection. The most highly selected capsid of the RC selection is, therefore, called hu.Hep.RC01, or RC01. As the cell type was the same for all capsids, for ease of reading they are only named with the platform and rank specification (i.e. RC01).

### Immunohistochemical Analysis

AAV capsids RC01, RC06, FT01, FT04, FT11, and NP59 were validated in more detail by injecting them individually into humanized FRG mice at a dose of 2×10¹¹ vgc. Livers were harvested and prepared for imaging two weeks after injection following previously published methods (Cabanes-Creus et al., 2020, Science Translational Medicine 2020 12(560): eaba3312).

### Example 2. Directed Evolution Using RC and FT Platforms in a Murine Xenograft Model of the Human Liver

Two different library selection approaches were utilized to select novel liver-tropic AAV capsids from a highly variable AAV library. The Replication-Competent (RC) platform is based on the wtAAV2 genome configuration and was successfully applied to develop novel AAV capsid variants (Grimm et al. 2008, Journal of Virology 2008 82(12): 5887; Lisowski et al. 2014, Nature 506: 38). Using this platform, AAV variants can be selected either by over-infection with a helper virus or PCR-amplification of DNA delivered to the target cells (Muller et al., 2003, Nat Biotechnol 21(9): 1040-1046; Dalkara et al., 2013, Sci Transl Med 5(189): 189ra176). However, it was hypothesized that due to the lack of control of the viral replication process, including target cell selection in complex tissues, and the inability to control the intra-cellular location of templates for PCR amplification, neither of these methods is a highly stringent or efficient way for selecting novel functional capsids. It was speculated that a platform where the cap gene would become the expressed transgene in the target cells could gear selection towards highly expressing vectors when combined with the directed evolution approach. Unfortunately, the RC platform cannot be used for selection based on cap gene expression, since the p40 promoter has low transcriptional activity in the presence of Rep proteins and absence of helper virus proteins and can also be inactive depending on the cell type. To overcome this, the original RC selection platform was re-engineered to introduce an exogenous promoter and a reporter gene.

In one variation, the resulting platforms contained a GFP expression cassette driven by a Spleen focus-forming virus (SFFV) long terminal repeat promoter (Faller et al., 1997, Journal of Cellular Physiology 172(2): 240-252) in reverse orientation to the p40-cap in place of the rep coding regions upstream of the p40 promoter. This system allows for selection of vector candidates based on functional transduction as measured by GFP expression and was, therefore, named Functional Transduction (FT) platform. Following transduction of target cells, this platform allows for sorting of eGFP-expressing cells followed by DNA extraction of vector genomes from these transduced cells (as previously described in Cabanes-Creus et al. 2020, Mol Ther Methods Clin Dev 17: 1139-1154). It was hypothesized also that the SFFV-p40 hybrid promoter may enable cap gene expression in the target cells, allowing for capsid recovery from RNA/cDNA.

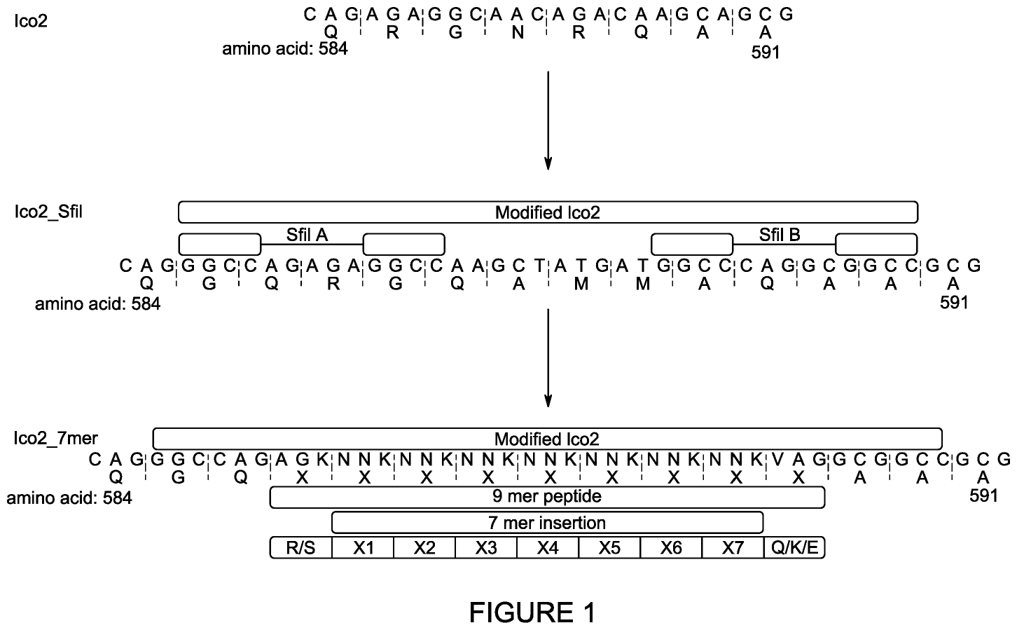

The above-described RC-Ad5 and FT platforms were used to identify novel liver-tropic capsids in a directed evolution selection process. To this end, an AAV2 peptide display library was built and inserted into RC and FT library constructs (see FIG. 1). Both libraries were used to perform AAV selection on primary human hepatocytes in a human liver xenograft model based on the Fah^{-/-}, Rag2^{-/-}, Il2rg^{-/-29} (FRG) mouse.

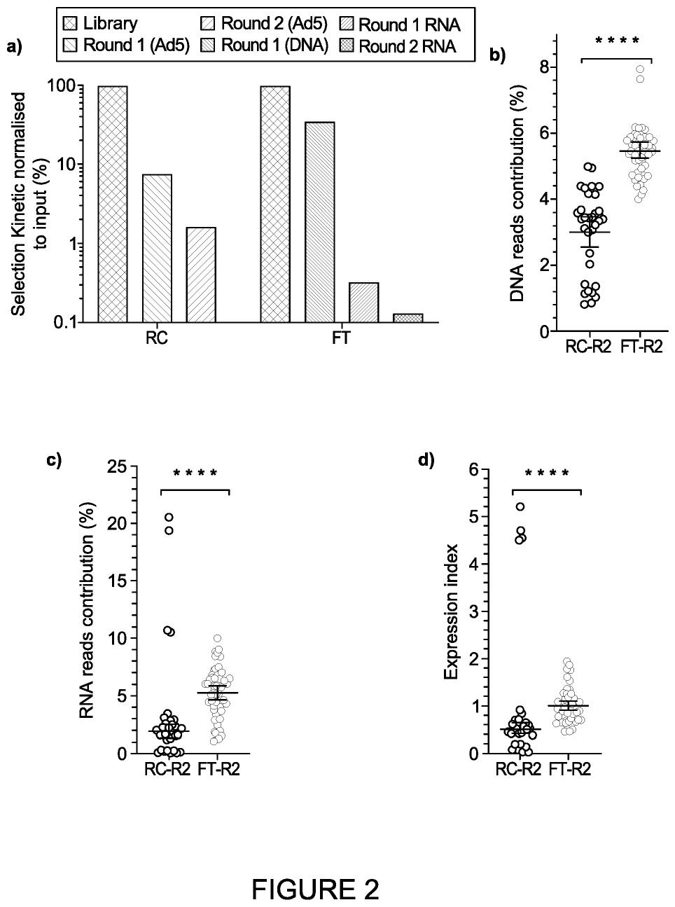

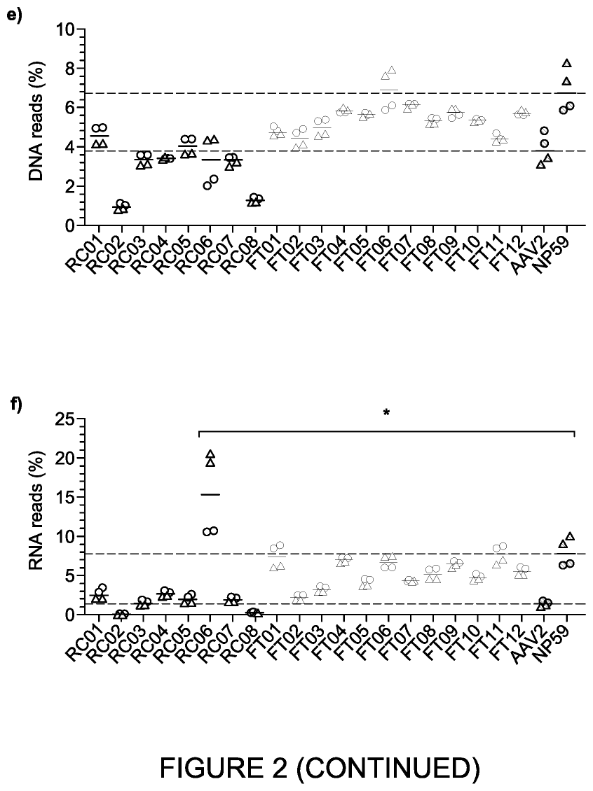

When using the RC platform, the total number of unique peptide variants in the library decreased from approximately 6.3×10⁵ (100%) to 4.7×10⁴ (7.45%) after the first round and to 1×10⁴ (1.6%) after the second round of selection (FIG. 2a), indicating a strong selection process. In comparison, the library in the FT platform analyzed at the DNA level contained 1.4×10⁶ (34.5%) unique peptide variants from an initial 4.2×10⁶ (100%) after one round of selection in GFP-positive sorted human hepatocytes. The selection process was more efficient when recovering capsid from RNA than from DNA. After one round of selection, only 1.4×10⁴ (0.3%) unique peptides were detected, which was further reduced to 5.5×10³ (0.1%) after the second round of selection (FIG. 2a). These selection kinetics indicate that, under these experimental conditions, the selection using RC-Ad5 was less stringent than the FT-RNA recovery.

The top 8 candidates from the RC-Ad5 selection (NGS read contribution above 1%) and the 12 top candidates from the FT RNA selection were vectorized. These were named RC01-RC08 and FT01-FT12. Table 3 shows the amino acid residues at each of the 9 peptide positions from FIG. 1 for each of the candidates. Table 4 provides the full (VP1) amino acid sequence of the each of the candidates.

All novel variants were used to package a barcoded ssAAV-LSP-GFP cassette (Cabanes-Creus et al. 2020, Science Translational Medicine 12(560): eaba3312; and Cabanes-Creus et al. 2020 Mol Ther Methods Clin Dev 17: 1139-1154). To exclude potential bias caused by the unique barcode, each vector was used to package three different barcoded cassettes. The AAV2 and strong human hepatocyte transducer AAV-NP59 were included as controls. All 22 vectors were mixed at 1:1:1 equimolar ratio and were injected intra-venously (2×10¹¹ vg/mouse) into four humanized FRG mice, two engrafted with hepatocytes from a male and two from a female donor. Analysis of the capsids recovered from the RC-Ad5 selection versus capsids identified using the FT-RNA selection, revealed that, on average, the capsids recovered from the FT-RNA selection performed significantly better at the level of cell entry (DNA) (FIG. 2b) and expression (RNA/cDNA) (FIG. 2c) in human hepatocytes. Most importantly, analysis of expression indices (RNA read contribution divided by DNA contribution) revealed that once FT-selected capsids enter human hepatocytes, they are significantly more efficient at contributing to RNA expression in these cells (FIG. 2d). These results show that the FT-RNA selection strategy is more stringent than RC-Ad5 and is more likely to lead to the identification of highly functional capsids.

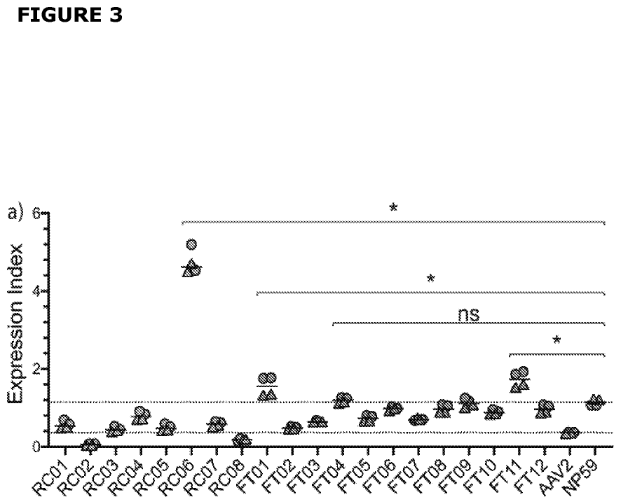

Interestingly, analysis of the performance of the individual 20 novel capsids revealed that the top variant selected using the RC-Ad5 strategy (AAV-RC01, 76% of all reads after 2^{nd} round of selection) was not a highly functional variant. Based on DNA entry (median: 4.6%) and RNA expression (median: 2.5%), AAV-RC01 was found to be slightly better than AAV2 (median: 3.8% and 1.4%, respectively, FIG. 2c-d). A surprising observation was that AAV-RC06, which was not efficient at cell entry (DNA, median: 3.3%), was efficient at driving RNA expression in both donor hepatocytes used (median: 15.3%), which was significantly higher than the RNA contribution of AAV-NP59 at a median of 7.8% of total RNA reads. Not surprisingly, AAV-RC06 also had the best expression index of all the capsids tested (median: 4.6, FIG. 3). This extremely strong variant also was represented by the outlier data points observed in the high RNA and expression index contribution in RC (FIG. 2c-d).

Capsids recovered from RNA using the FT selection platform were more efficient at entering cells compared to AAV2, with AAV-FT06 (median: 6.9%) being even slightly more efficient than AAV-NP59 (median: 6.7%, FIG. 3e). At the RNA level, four candidates (AAV-FT01, AAV-FT04, AAV-FT06, and AAV-FT11) were found to be comparable to AAV-NP59 (FIG. 2f), with three of those able to achieve higher expression indices (medians: AAV-FT01: 1.6; AAV-FT04: 1.2; AAV-FT11: 1.7) than AAV-NP59 (median: 1.1, FIG. 3).

As a final validation of human hepatocyte specificity, the performance of the most functional novel capsids (AAV-RC06, AAV-FT01, AAV-FT04, and AAV-FT11) as well as AAV-RC01 were evaluated in individual animals using immunohistochemical (IHC) analysis of transgene expression. AAV-NP59 was used as a positive control. It was found that all novel peptide variants had the same level of specificity to human hepatocytes as AAV-NP59 given that all GFP-positive cells were also positive for the human cell marker glyceraldehyde 3-phosphate dehydrogenase (data not shown). Moreover, as expected, the variant AAV-RC01 showed the lowest level of GFP expression, which was especially evident when examining the chimeric liver using UV light prior to sectioning (data now shown). However, IHC revealed that AAV-NP59 led to the strongest GFP expression of all the variants tested, an unexpected result given the NGS data for GFP RNA and the expression index data. However, the unexpectedly high GFP expression observed for AAV-NP59 might be attributable to the fact that the FRG mouse injected with AAV-NP59 had a lower level of human cells engraftment, as evident by smaller size of the human clusters when compared to mice treated with other vectors (data now shown). Analysis of serum obtained from the mouse the day of vector administration further confirms lower engraftment with a human albumin level of 0.73 mg/mL in the AAV-NP59 mouse compared to other animals with human albumin levels ranging from 0.77-4.43 mg/mL. Thus, compared to other vectors tested in this study, AAV-NP59 was effectively injected at a higher vector dose per human hepatocytes.

TABLE 3
Amino acids at 9mer peptide

| Position | 1 | 2 | 3 | 4 | 5 | 6 | 7 | 8 | 9 |
|---|---|---|---|---|---|---|---|---|---|
| Possibility | R/S | X1 | X2 | X3 | X4 | XS | X6 | X7 | Q/E/K |
| AAV2-RC01 | S | T | T | H | L | S | P | P | Q |
| AAV2-RC02 | S | E | L | E | E | M | N | N | K |
| AAV2-RC03 | S | P | S | S | P | A | P | A | Q |
| AAV2-RC04 | R | P | E | T | Q | A | K | P | Q |
| AAV2-RC05 | S | T | A | Y | T | P | A | P | Q |
| AAV2-RC06 | R | S | Q | R | E | T | V | W | K |
| AAV2-RC07 | S | V | M | M | V | G | G | R | E |
| AAV2-RC08 | R | K | D | P | E | V | S | E | Q |
| AAV2-FT01 | S | K | T | N | L | D | R | A | Q |
| AAV2-FT02 | S | A | T | Q | P | Y | A | S | Q |
| AAV2-FT03 | R | T | S | A | H | Q | V | G | E |
| AAV2-FT04 | R | S | P | K | G | S | A | W | E |
| AAV2-FT05 | S | P | Q | Q | P | P | R | S | Q |
| AAV2-FT06 | S | S | R | T | P | Q | H | K | Q |
| AAV2-FT07 | S | P | R | H | P | V | T | T | Q |
| AAV2-FT08 | S | H | T | T | G | S | P | A | K |
| AAV2-FT09 | R | M | T | G | K | T | G | Y | E |
| AAV2-FT10 | R | Q | A | T | S | G | F | D | K |
| AAV2-FT11 | R | A | K | E | M | R | S | E | Q |
| AAV2-FT12 | S | A | F | S | Q | S | A | V | K |

TABLE 4
Capsid Sequences (VP1 protein)

| SEQ | | |
|---|---|---|
| ID | Capsid | Capsid sequence |
| NO | name | (sequence corresponding to 9mer peptide insert in bold) |
| 1 | AAV2 | MAADGYLPDWLEDTLSEGIRQWWKLKPGPPPPKPAERHKDDSRGLVLPGYKYLGPFNGLD |
| | prototypic | KGEPVNEADAAALEHDKAYDRQLDSGDNPYLKYNHADAEFQERLKEDTSFGGNLGRAVFQ |
| | capsid-VP1 | AKKRVLEPLGLVEEPVKTAPGKKRPVEHSPVEPDSSSGTGKAGQQPARKRLNFGQTGDAD |
| | (protein) | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVPANPSTTFSAA |
| | | KFASFITQYSTGQVSVEIEWELQKENSKRWNPEIQYTSNYNKSVNVDFTVDTNGVYSEPRP |
| | | IGTRYLTRNL |
| 2 | AAV2-RC01 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSTTHLSPPQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 3 | AAV2-RC02 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSELEEMNNKQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 4 | AAV2-RC03 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSPSSPAPAQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 5 | AAV2-RC04 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRPETQAKPQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 6 | AAV2-RC05 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSTAYTPAPQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 7 | AAV2-RC06 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRSQRETVWKQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 8 | AAV2-RC07 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSVMMVGGREQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 9 | AAV2-RC08 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRKDPEVSEQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 10 | AAV2-FT01 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSKTNLDRAQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 11 | AAV2-FT02 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSATQPYASQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 12 | AAV2-FT03 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRTSAHQVGEQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 13 | AAV2-FT04 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRSPKGSAWEQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 14 | AAV2-FT05 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSPQQPPRSQQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 15 | AAV2-FT06 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSSRTPQHKQQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 16 | AAV2-FT07 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSPRHPVTTQQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 17 | AAV2-FT08 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSHTTGSPAKQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 18 | AAV2-FT09 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRMTGKTGYEQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 19 | AAV2-FT10 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRQATSGFDKQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |
| 20 | AAV2-FT11 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRAKEMRSEQQAAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 21 | AAV2-FT12 | MAADGYLPDWLEDTLSEGIRQWWKLKPGPPPPKPAERHKDDSRGLVLPGYKYLGPFNGLD |
| | | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSAFSQSAVKQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL |

1. An AAV capsid polypeptide, comprising a peptide modification relative to the AAV2 capsid polypeptide set forth in SEQ ID NO:1, wherein:
the peptide modification is in variable region 8 (VRVIII) and;
the peptide modification comprises a 7 amino insertion relative to the AAV2 capsid polypeptide set forth in SEQ ID NO:1, and comprises the sequence set forth in any one of SEQ ID Nos:22-41; and
the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to positions 1-735, 138-735 or 203-735 of SEQ ID NO:1.

2. The AAV capsid polypeptide of claim 1, wherein the peptide modification is in the region of the capsid polypeptide spanning positions 585-589, with numbering relative to SEQ ID NO:1.

3. The AAV capsid polypeptide of claim 1 or 2, wherein the peptide modification comprises a 7 amino insertion after position 587 relative to the AAV2 capsid polypeptide set forth in SEQ ID NO:1.

4. The AAV capsid polypeptide of claim 3, wherein the peptide modification comprises amino acid substitutions at positions 587 and 588 relative to the AAV2 capsid polypeptide set forth in SEQ ID NO:1.

5. The AAV capsid polypeptide of any one of claims 1-4, comprising one or more amino acid substitutions at position 585, 586, and/or 589 relative to the AAV2 capsid polypeptide set forth in SEQ ID NO:1.

6. The AAV capsid polypeptide of claim 5, wherein the amino acid substitution is R585G, G586Q and/or Q589A, relative to the AAV2 capsid polypeptide set forth in SEQ ID NO:1.

7. The AAV capsid polypeptide of any one of claims 1-6, comprising the sequence of amino acids set forth in any one of SEQ ID NOs:2-21, the sequence of amino acids set forth as amino acids 138-742 of any one of SEQ ID NOs:2-21, or the sequence of amino acids set forth as amino acids 203-742 of any one of SEQ ID NOs:2-21; or a sequence having at least or about 85%, 86%, 87%, 88%, 89%, 90%, 91%, 92%, 93%, 94%, 95%, 96%, 97%, 98%, or 99% sequence identity thereto.

8. An AAV capsid polypeptide, comprising:
a) a VP1 protein comprising the sequence of amino acids set forth in any one of SEQ ID NOs:2-21;
b) a VP2 protein comprising the sequence of amino acids set forth as amino acids 138-742 of any one of SEQ ID NOs:2-21;
c) a VP3 protein comprising the sequence of amino acids set forth as amino acids 203-742 of any one of SEQ ID NOs:2-21, or
d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c), wherein the capsid polypeptide comprises, at positions 587-595 with numbering relative to any one of SEQ ID NOs:2-21, the peptide sequence set forth in any one of SEQ ID Nos:22-41.

9. An AAV vector, comprising the capsid polypeptide of any one of claims 1-8.

10. The AAV vector of claim 8, wherein the vector exhibits increased transduction efficiency of human hepatocytes compared to an AAV vector comprising a capsid polypeptide comprising the sequence of amino acids set forth in SEQ ID NO:1.

11. The AAV vector of claim 10, wherein transduction efficiency is increased by at least or about 10%, 20%, 30%, 40%, 50%, 60%, 70%, 80%, 90%, 100%, 200%, 300%, 400% or 500%.

12. The AAV vector of any one of claims 9-11, further comprising a heterologous coding sequence.

13. The AAV vector of claim 12, wherein the heterologous coding sequence encodes a peptide, polypeptide or polynucleotide.

14. The AAV vector of claim 13, wherein peptide, polypeptide or polynucleotide is a therapeutic peptide, polypeptide or polynucleotide.

15. An isolated nucleic acid molecule encoding the capsid polypeptide of any one of claims 1-8.

16. A vector comprising the nucleic acid molecule of claim 15.

17. The vector of claim 16, wherein the vector is selected from among a plasmid, cosmid, phage and transposon.

18. A host cell, comprising the AAV vector of any one of claims 9-14, the nucleic acid molecule of claim 15, or the vector of claim 16 or claim 17.

19. A method for introducing a heterologous coding sequence into a host cell, comprising contacting a host cell with the AAV vector of any one of claims 12-14.

20. The method of claim 19, wherein the host cell is a hepatocyte.

21. The method of claim 19 or 20, wherein contacting a host cell with the AAV vector comprises administering the AAV vector to a subject.

22. The method of claim 21, wherein administration of the AAV vector to the subject effects treatment of a liver-associated disease or condition.

23. The method of claim 19 or 20, wherein the method is in vitro or ex vivo.

24. A method for producing an AAV vector, comprising culturing a host cell comprising a nucleic acid molecule encoding the capsid polypeptide of any one of claims 1-8, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide of any one of claims 1-8, wherein the capsid encapsidates the heterologous coding sequence.

25. The method of claim 24, wherein the host cell is a hepatocyte.

26. Use of the AAV vector of any one of claims 9-14 for the preparation of a medicament for treating a liver-associated disease or condition.
